(19) United States
(12) Patent Application Publication
(10) Pub. No.: US 2026/0240062 A1
(43) Pub. Date: Aug. 20, 2026

(54) DIGGING TOOL

(71) Applicant: Christopher Lee Engelbrecht, Paxton, IL (US)

(72) Inventor: Christopher Lee Engelbrecht, Paxton, IL (US)

(21) Appl. No.: 19/541,108

(22) Filed: Feb. 16, 2026

Related U.S. Application Data
(60) Provisional application No. 63/759,396, filed on Feb. 17, 2025.

(51) Int. Cl. A01B 1/02; A61B 17/3201

(52) U.S. Cl. CPC A01B 1/02; A61B 17/3201

(57) ABSTRACT

A digging tool comprising a handle, an extension, and a blade is disclosed. The handle has a first end and a second end and extends along a first axis. The extension may have a main portion and first and second transition pieces. The first transition piece extends from the second end of the handle to the main portion. The main portion extends between the first transition piece and the second transition piece along a second axis generally perpendicular to the first axis. The second transition piece extends from the main portion to the blade. The blade may be generally planar and may comprise a body and a curved portion, terminating in a distal tip. The body of the blade may extend along a third axis, generally parallel to the first axis. The blade may transition from the body to the curved portion at an inflection region.

### CROSS-REFERENCE TO RELATED APPLICATIONS

This application claims the benefit of and priority to United States Provisional Patent Application Serial No. 63/759,396 filed February 17, 2025, the disclosure which is herein incorporated by reference in its entirety.

### FIELD OF THE INVENTION

The present disclosure relates to a novel and advantageous digging tool Particularly, the present disclosure relates to a novel and advantageous digging tool comprising a handle, an extension, and a blade. More particularly, the present disclosure relates to novel and advantageous digging tool comprising a handle, an extension, and a blade, wherein the handle and a body of the blade are parallel to one another and a main portion of the extension is perpendicular thereto.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Various hand tools have been used in gardening, including hand rakes, trowels, and the like. The present disclosure includes improvements over the current state of the art.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

In accordance with some embodiments, the disclosure provides a digging tool. In various embodiments, the digging tool includes a handle, an extension, and a blade. The handle has a first end and a second end and extends along a first axis. The extension may have a main portion and first and second transition pieces. The first transition piece extends from the second end of the handle to the main portion. The main portion extends between the first transition piece and the second transition piece along a second axis generally perpendicular to the first axis. The second transition piece extends from the main portion to the blade. The blade may be generally planar and may comprise a body and a curved portion, terminating in a distal tip. The body of the blade may extend along a third axis, generally parallel to the first axis. The blade may transition from the body to the curved portion at an inflection region.

In various embodiments, the inflection region where the blade transitions to the curved distal portion may be located about half to two thirds of the length of the blade toward the distal tip. The blade can be pentagonal in shape with five edges meeting at corners, or have more or less sides, or be defined by a continuously curved perimeter, or a perimeter formed by a plurality of sides with rounded corners where the sides meet. In some embodiments, the blade can include a “V” shaped leading edge terminating in the distal tip.

In some embodiments, the handle can be defined by a length L1, the extension can be defined by a length L2, and the blade can be defined by a length L3, wherein the lengths L1, L2 and L3 can be of approximately the same length, or of different lengths. The main portion of the extension can be oriented orthogonally, or obliquely with respect to one or both of the first transition piece and second transition piece of the extension.

The disclosure further provides methods of using the tools set forth herein. In one illustrative method, the method can include using a tool as described herein by inserting the distal tip of the tool into soil to create a small initial fracture in the soil and to center the tool into a path of intended use, inserting the curved, distal section of the blade into the soil, inserting the flat, proximal section of the blade into the soil, and uplifting the tool from the soil to remove and displace the soil.

It is to be understood that both the foregoing general description and the following detailed description are exemplary and are intended to provide further explanation of the embodiments disclosed herein.

The accompanying drawings, which are incorporated in and constitute part of this specification, are included to illustrate and provide a further understanding of the method and system of the disclosure. Together with the description, the drawings serve to explain the principles of the disclosed embodiments.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 depicts an isometric side view of an illustrative digging tool in accordance with the present disclosure.

FIG. 2 depicts a partial, top perspective view of the tool of FIG. 1.

### DETAILED DESCRIPTION

The present disclosure is generally directed to garden tools, and particularly toward embodiments of smaller garden tools that can be operated conveniently by a single hand of the user.

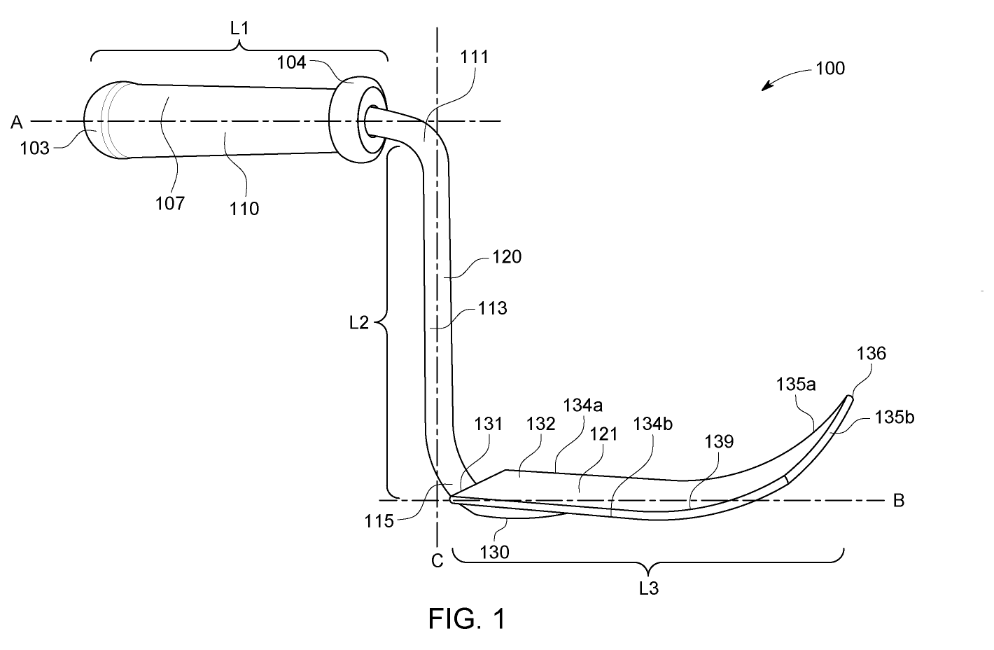

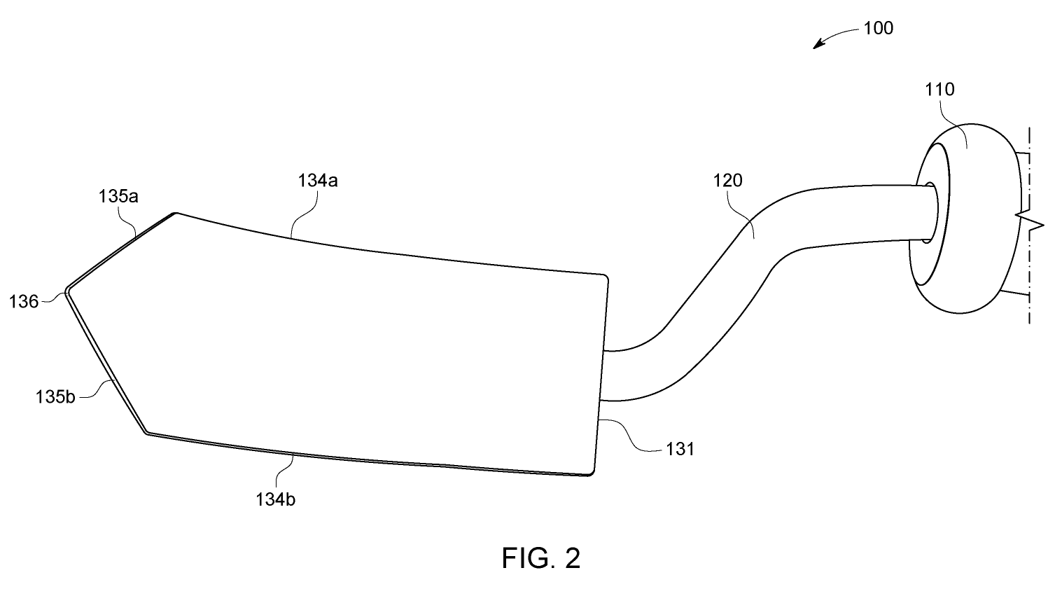

For purposes of illustration, and not limitation, FIGS. 1 and 2 illustrate a digging tool in accordance with the present disclosure. The geometry of the digging tool accounts for the natural movement of a user’s elbow and shoulder, and reduces required wrist movement, and also amplifies any movement of the wrist.

The digging tool comprises a handle 110, an extension 120, and a blade 130. In the embodiment shown, the handle 110 is provided along Axis A, a main portion of the extension 120 is provided along Axis B, and a body of the blade 130 is provided along Axis C. Axis A and Axis C may be generally parallel, with Axis B being generally perpendicular thereto. In some embodiments, Axis A and Axis C may be offset by approximately 4-12 inches.

The handle 110 has a first end 103, a second end 105, and a body 107 extending therebetween. The body 107 has a diameter large enough to fit the user’s hand. The handle 110 may be formed of any material or any blend of materials. For example, the handle may be wood, plastic, composite, or other. The handle may have a unitary material composition, or may have discrete section comprising different materials. In some embodiments, a grip portion of the handle may be or include wood and the grip portion may be provided about a structural support that is metal. The handle may have a length L1 of approximately 3-8 inches.

The extension rod 120 extends between the handle 110 and the blade 130. The extension rod 120 may include a main portion 113, a first transition piece 111, and a second transition piece 115. The first transition piece 111 extends from the second end 105 of the handle 110 to the main portion 113 and may be curved. The main portion 113, the portion provided along Axis C, is generally perpendicular to the first transition piece 111. The second transition piece 115 extends from the main portion 113 to the blade 130 and may be curved. In some embodiments, the first transition piece may range from 1-3 inches in length, the main portion may range from 4-12 inches in length, and the second transition piece may range from 1-3 inches in length. In other embodiments, the first transition piece 111 and/or the second transition piece 115 may be omitted. The extension rod 120 may have a length L2 of approximately 4-12 inches.

The blade 130 may comprise a body 121 and a curved portion 123. The blade is generally planar, in that its total length and width dimension substantially exceed its thickness. The body 121 may be coupled to the second transition piece 115 of the extension rod 120. In some embodiments, the body 121 may partially overlap the second transition piece 115 of the extension 120. The curved portion 123 is provided at a distal region of the blade 130. The curved portion 123 may extend from an inflection region 139 to a distal tip 136. The inflection region 139 may be provided about one half to two thirds of the way along a length of the blade 130. In some embodiments, the blade may have a length L3 of approximately 4-12 inches, with the body ranging from 1-8 inches in length and the curved portion ranging from 3-8 inches in length. In some embodiments, the curved portion 123 may curve form the body 121 at a curvature approximating an angle of approximately 15-45 degrees.

The main portion 113 of the extension 120 can be perpendicular to the handle 110 and the blade 130, with the transition piece 111 curving from the plane of the handle 110 to the plane of the main portion 113 and the transition piece 115 curving from the plane of the main portion 113 to the plane of the body 121 of the blade 130. The distance from the handle 110 to the blade 130 is not critical. However having the distance too short may reduce the effectiveness of the action of the tool and having the distance too long may create a large fulcrum that makes using the tool difficult to use if the user has a reduced strength.

As shown in FIG. 2, the blade 130 may be pentagonal in shape with five edges (131, 134a, 134b, 135a, 135b). In other embodiments, the blade may have a different shape and may have more or fewer sides, may be defined by a continuously curved perimeter, or may be defined a perimeter formed by a plurality of sides with rounded corners where the sides meet. In some embodiments, the blade can include a “V” shaped leading edge 135, 135 terminating in the distal tip 136.

The disclosure further provides methods of using tools such set forth herein. In one illustrative method, the method can include using the tool of FIGS. 1 and 2 by inserting a distal tip of the blade of the tool into soil to create a small initial fracture in the soil and to center the tool into a path of intended use. The curved, distal section of the blade may be inserted into the soil, followed by at least a portion of the flat, proximal section of the blade. The tool may then be lifted to remove and displace soil at or near the insertion point.

The blade 130 is preferably in a plane that is parallel to a central axis of the handle 110. The illustrated handle to blade placement provides for a neutral placement of the users wrist to avoid or reduce stress injury. The placement also provides a better grip angle for users with arthritis or reduced grip strength. Having a user’s wrist in a neutral location allows for any movement of the wrist to increase the action of the blade 130 of the tool. As illustrated, the blade 130 has a central point 136 to create a small initial fracture in soil and to naturally center the tool into the path of intended use so that rocks or other debris do not deflect the tool 100 into an unwanted path. The distal curvature of the blade 130 allows the tool to use the weight of the soil to bring the cutting surface into and across the soil. The blade 130 further has a flat area 132 proximate the second transition piece 115 for soil to rest upon while being uplifted and displaced during the motion of the tool 100.

The devices and methods disclosed herein can be used for other purposes as illustrated or can be modified as needed to suit the particular application. In view of the many possible embodiments to which the principles of this disclosure may be applied, it should be recognized that the illustrated embodiments are only preferred examples of the disclosure and should not be taken as limiting the scope of the disclosure.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A tool, comprising:
a handle having a first end and a second end, the handle extending along a first axis;
an extension rod having a main portion and first and second transition pieces, wherein the main portion extends along a second axis generally perpendicular to the first axis; and
a blade comprising a body and a curved portion, the curved portion terminating in a distal tip, wherein the body extends along a third axis generally parallel to the first axis.

2. The tool of claim 1, wherein the blade transitions form the body to the curved portion at an inflection point.

3. The tool of claim 2, wherein the inflection point is at approximately 2/3 of a length of the blade towards the distal tip.

4. The tool of claim 2, wherein the inflection point is at approximately 1/2 of a length of the blade.

5. The tool of claim 1, wherein at least the blade body is generally planar.

6. The tool of claim 1, wherein the blade is pentagonal in shape.

7. The tool of claim 1, wherein the blade includes a “V” shaped leading edge terminating in the distal tip.

8. The tool of claim 1, wherein the handle is defined by a length L1, the extension rod is defined by a length L2, and the blade is defined by a length L3, wherein the lengths L1, L2 and L3 are of approximately the same length.

9. The tool of claim 1, wherein the first and second transition pieces are curved.

10. A method of using the tool of claim 1, comprising:
inserting the distal tip of the tool into soil to create a small initial fracture in the soil and to center the tool into a path of intended use;
inserting the curved, distal section of the blade into the soil;
inserting the flat, proximal section of the blade into the soil; and
uplifting the tool from the soil to remove and displace the soil.

11. A tool, comprising:
a handle having a first end and a second end, the handle extending along a first axis;
an extension rod having a main portion, wherein the main portion extends along a second axis generally perpendicular to the first axis; and
a blade comprising a body, wherein the body extends along a third axis generally parallel to the first axis, and wherein the blade is configured to create a small initial fracture in soil.

12. The tool of claim 11, wherein the extension rod includes a first extension piece coupling the main portion to the handle.

13. The tool of claim 12, wherein the extension rod includes a second extension coupling the main portion to the blade.

14. The tool of claim 13, wherein the first and second transition pieces are curved.

15. The tool of claim 11, wherein the blade include a curved portion, the curved portion terminating in a distal tip.

16. The tool of claim 15, wherein the blade includes a “V” shaped leading edge terminating in the distal tip.

17. The tool of claim 11, wherein in the first axis is substantially parallel to the third axis.

18. The tool of claim 11, wherein the blade body includes a flat area 132 adjacent the proximal end of the blade.
